(19) United States
(12) Patent Application Publication
(10) Pub. No.: US 2011/0224435 A1
(43) Pub. Date: Sep. 15, 2011

(54) PROCESS FOR PREPARATION OF AMORPHOUS LOPINAVIR

(75) Inventors: Sujoy Biswas, Nadia (IN); Ramendra Singh Rathore, Farrukhabad (IN); Keya Karanjai, Varanasi (IN); Chandra Has Khanduri, Gurgaon (IN)

(73) Assignee: RANBAXY LABORATORIES LIMITED, New Deli (IN)

(21) Appl. No.: 12/672,104

(22) PCT Filed: Aug. 6, 2008
(86) PCT No.: PCT/IB2008/053167

§ 371 Date: Apr. 21, 2010

(30) Foreign Application Priority Data
Aug. 7, 2007 (IN) 1672/DEL/2007

(51) Int. Cl. C07D 239/22

(52) U.S. Cl. 544/316

(57) ABSTRACT

The present invention relates to a process for preparation of amorphous lopinavir, which is HIV protease inhibitor of Formula (I). Using agitated film drying.

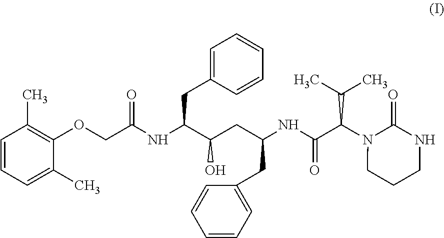

### FIELD OF THE INVENTION

The present invention relates to processes for the preparation of amorphous lopinavir, an HIV protease inhibitor.

### FIELD OF THE INVENTION

Lopinavir of Formula I is chemically [1S-[1R*,(R*),3R*,4R*]]-N-[4-[[(2,6-dimethyl-phenoxy)acetyl]amino]-3-hydroxy-5-phenyl-1-(phenylmethyl)pentyl]tetrahydro-alpha-(1-methylethyl)-2-oxo-1(2H)-pyrimidineacetamide and is indicated in combination with other antiretroviral agents for the treatment of HIV-infection.

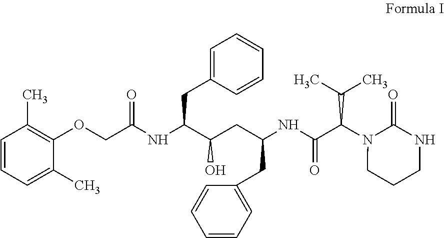

U.S. Pat. No. 5,914,332 provides a process for preparing amorphous lopinavir which involves dissolving lopinavir in an organic solvent (for example, ethanol, isopropanol, acetone, or acetonitrile) and then adding the solution to water. For example, lopinavir is dissolved in ethanol (from about 2 to about 4 mL/g) and the ethanolic solution is added with stirring to water (from about 10 about 100 mL/g) to provide amorphous lopinavir. However, this process for the preparation of amorphous lopinavir is not effective on the kilogram scale and thus is not commercially suitable.

PCT Publication No. WO 01/074787 provides various crystalline Forms (Types I, II, III, IV) of solvated and non-solvated lopinavir. It further provides a process for the preparation of amorphous lopinavir which involves dehydration/desolvation of Type I hydrated crystal form/Type II solvated crystal forms.

PCT Publication Nos WO 2006/100552 and WO 2006/090264 provide process for the preparation of crystalline lopinavir.

Organic Process Research & Development, 3, 145-148 (1999), and Organic Process Research & Development, 4, 264-269 (2000); provide a crystallization process for the preparation of crystalline lopinavir which involves recrystallization from mixtures of ethyl acetate and heptane. However, the crystalline lopinavir obtained contains small amounts of solvents and removal of the final traces of solvents proved exceedingly difficult, and even extensive drying after milling (to reduce particle size) did not facilitate its complete removal. It further provides the crystallized product obtained contains appromixately 2% residual ethyl acetate which cannot be removed by further drying.

There is need in the art for new methods for preparing amorphous lopinavir.

### SUMMARY OF THE INVENTION

In one aspect, the present invention provides a process for preparing amorphous lopinavir comprising removing solvent from a solution comprising lopinavir using agitated thin film drying.

In another aspect, the present invention provides a process for drying lopinavir wherein the said process comprises:

a) feeding a solution or a slurry of lopinavir into an agitated thin film dryer (ATFD),

b) drying the fed lopinavir by agitated thin film drying, and

c) collecting dry lopinavir from the agitated thin film dryer.

### BRIEF DESCRIPTION OF THE DRAWING

FIG. 1 is an XRPD of lopinavir prepared by agitated thin film drying.

Powder XRD of the samples were determined by using X-Ray Difractometer, Rigaku Corporation, RU-H3R, Goniometer CN2155A3, X-Ray tube with Cu target anode, Divergence slits 1 0, Receiving slit 0.15 mm, Scatter slit 1°, Power: 40 KV, 100 mA, Scanning speed: 2 deg/min step: 0.02 deg, Wave length: 1.5406 A.

### DETAILED DESCRIPTION OF THE INVENTION

In one aspect, the present invention provides a process for preparing amorphous lopinavir comprising removing solvent from a solution comprising lopinavir using agitated thin film drying.

In another aspect, the present invention provides a process for drying lopinavir wherein the process comprises:

a) feeding a solution or a slurry of lopinavir into an agitated thin film dryer (ATFD),

b) drying the fed lopinavir by agitated thin film drying, and

c) collecting dry lopinavir from the agitated thin film dryer.

The starting material lopinavir may be prepared according to the processes known to those of skill in the art. One such process is provided in U.S. Pat. No. 5,914,332.

A solution or slurry of lopinavir is prepared by mixing lopinavir with an organic solvent. The organic solvent can be selected from, for example, methanol, ethanol, isopropanol, tetrahydrofuran, acetone, acetonitrile and the like. The solution or slurry is fed into an agitated thin film dryer (ATFD). The bath temperature, feed rate and speed of the ATFD rotor can be adjusted to optimize the output and particle size distribution.

The bath temperature is preferably maintained between about 60-100° C. The feed rate is set between about 10 ml/10 minutes and 100 ml/10 minutes. The set feed rate is preferably constant for the whole process. The speed of the rotor can be set between about 60-160 revolutions per minute.

The drying process is accompanied by the application of vacuum. The drying process is carried at about 60-100° C. and for sufficient time to effect maximum removal of the solvents and then cooled to room temperature and unloaded. The solid obtained was dried under vacuum at about 60-100° C. for about 8 to 15 hours to provide amorphous lopinavir.

While the present invention has been described in terms of its specific embodiments, certain modifications and equivalents will be apparent to those skilled in the art and are intended to be included within the scope of the present invention.

### Example 1

### Preparation of Amorphous Lopinavir

To stirred methanol (180 ml) was added lopinavir (60 g) at 25°-30° C. Stiffing was continued for 15-20 minutes to get a clear solution. The methanolic solution was fed into a Rotavapor over a period of 2-2.5 hours with the following settings: bath temperature: 70-75° C.; Feeding rate: 20 ml/10 minutes; and Vacuum (740-750 mm Hg and RPM 100-120).

After completion of feeding, the mass was kept under vacuum (740-750 mm Hg) at 70-75° C. for 45-60 minutes and then cooled to room temperature and unloaded. The solid material was dried under vacuum at 65-70° C. for 10-12 hours to provide amorphous lopinavir, in a yield of 54 g.

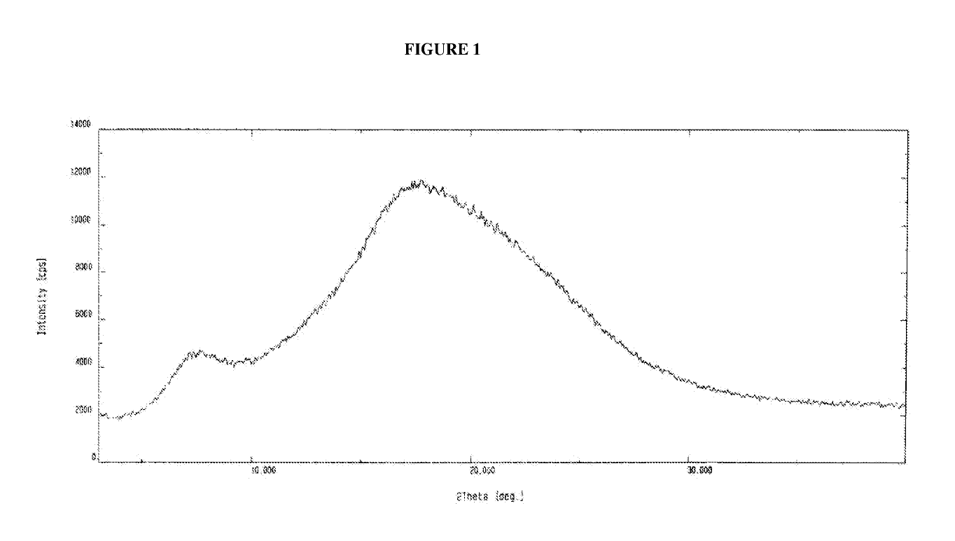

The sample was analysed by X-Ray Powder Diffraction (XRPD). Amorphous lopinavir was obtained, as demonstrated in FIG. 1.

1. A process for preparing amorphous lopinavir comprising removing solvent from a solution comprising lopinavir using agitated thin film drying.

2. A process for drying lopinavir wherein the process comprises:
a) feeding a solution or a slurry of lopinavir into an agitated thin film dryer (ATFD),
b) drying the fed lopinavir by agitated thin film drying, and
c) collecting dry lopinavir from the agitated thin film dryer.

3. A process according to claim 2, wherein the solution or slurry of lopinavir is prepared by mixing lopinavir with an organic solvent.

4. A process according to claim 3, wherein the organic solvent is selected from methanol, ethanol, isopropanol, tetrahydrofuran, acetone, acetonitrile.

5. A process according to claim 2, wherein the feed rate is controlled between about 10 ml/10 minutes and 100 ml/10 minutes.

6. A process according to claim 2, wherein the drying is accompanied by the application of vacuum at a temperature of about 60-100° C.

7. A process according to claim 2, wherein the lopinavir is collected at step c) as an amorphous powder.
